(19) United States
(12) Patent Application Publication
(10) Pub. No.: US 2009/0112252 A1
(43) Pub. Date: Apr. 30, 2009

(54) CATHETER WITH REMOVABLE FILTER RETRIEVAL TIP

(75) Inventor: Brendan CASEY, Barna (IE)

Correspondence Address: Sughrue Mion-ABBOTT LABS, 2100 Pennsylvania Avenue, N.W., Washington, DC, 20037, US

(73) Assignee: SALVIAC LIMITED, Dublin 2 (IE)

(21) Appl. No.: 11/925,823

(22) Filed: Oct. 27, 2007

(51) Int. Cl. A61M 29/00

(52) U.S. Cl. 606/200

(57) ABSTRACT

A removable retrieval tip for use with a catheter, a catheter tip, and a system for retrieval of an embolic filter device are provided. The removable retrieval tip includes a filter reception space for receiving a filter; and an interface portion for interfacing with the catheter. The catheter tip for receiving a removable retrieval tip includes a catheter body having an inside wall and an outside wall; an inflatable balloon disposed on the outside wall of the catheter body; and at least two indentations which are spaced from a distal end of the catheter tip and are formed in the inside wall of the catheter body. The system includes a catheter having a catheter tip at a distal end thereof, and a removable retrieval tip including a cylindrical body and an interface portion provided at a proximal end of the cylindrical body for interfacing with the catheter tip.

### BACKGROUND OF THE INVENTION

1. Field of the Invention

Apparatuses and systems consistent with the present invention relate to catheters used in treating buildup of plaque and other occlusions in blood vessels and, more particularly, to a angioplasty balloon catheter which includes a removable filter retrieval tip.

2. Description of the Related Art

Arteriosclerosis, also known as atherosclerosis, is a common human ailment arising from the deposition of fatty-like substances, referred to as atheroma or plaque, on the walls of blood vessels. Such deposits occur in peripheral blood vessels that feed limbs of the body, coronary blood vessels that feed the heart, and in carotid blood vessels that feed the head, neck, and brain. Localized accumulation of deposits within regions of the blood vessels may result in stenosis, or narrowing of the vascular channel. When this occurs, blood flow is restricted and the person's health is at serious risk.

Numerous approaches for reducing and removing such vascular deposits have been proposed, including balloon angioplasty, in which a balloon-tipped catheter is used to dilate a stenosed region within the blood vessel; atherectomy, in which a blade or other cutting element is used to sever and remove the stenotic material; laser angioplasty, in which laser energy is used to ablate at least a portion of the stenotic material; and the like. Related art angioplasty catheters are well known for their utility in treating the build-up of plaque and other occlusions in blood vessels.

A related art catheter is used to carry a dilatation balloon to a treatment area, where fluid under pressure is supplied to the balloon, to expand the balloon against an obstruction. The expansion of the balloon may also deploy a stent or other treatment device in the treatment area. Examples of related art angioplasty catheters are provided in U.S. Pat. No. 6,712,783 and in U.S. Patent Application Publication No. 2007/0016240, the disclosures of which are incorporated herein by reference.

In the case of treating the buildup of plaque or other occlusions in the carotid artery, a related art procedure commonly uses a filter element which is deployed downstream of a treatment area, i.e., between the treatment area and the brain, in order to filter and remove any pieces of plaque or occlusion material which may be dislodged during treatment and thus enter the brain causing a stroke or other damage. U.S. Patent Application Publication No. 2002/0049467, which is herein incorporated by reference in its entirety, describes such a related art procedure.

First, a delivery catheter is loaded with the filter element. The delivery catheter is then inserted through the leg and threaded, typically using a guidewire, to the treatment region. The delivery catheter is then advanced through the region to be treated and the filter element is deployed downstream of the treatment region, as indicated above. The delivery catheter is then removed from the body. A treatment catheter, such as, for example, an angioplasty balloon catheter, is inserted through the leg and threaded through the vasculature to the treatment region in order to carry out the treatment. Thus, the angioplasty balloon is inflated at the stenosed region, and the filter element filters any dislodged plaque material. The treatment catheter is then removed from the body, and a retrieval catheter is inserted through the leg and threaded through the vasculature to the treatment region. The removal catheter is then carefully advanced through the treatment region, which may also have a stent in place, to the filter element. The filter element is retrieved into the retrieval catheter, and the retrieval catheter, along with the filter element, is removed from the body.

Such related art catheters present a number of problems. One such problem is that there are a number of different types and/or designs of filter elements. Various filter elements may operate slightly differently and have different features, thus having different removal needs. As such, various filter elements require different designs of retrieval catheters in order for the filter element to be removed effectively. Physicians may therefore be required to have multiple filter elements and multiple retrieval catheters, which are suited to the filter element used, available depending on the treatment desired. This results in an increased cost in terms of maintaining a large amount of equipment and of treating the patient.

Another problem with the related art catheters is that the procedure for deploying the filter element, treating the treatment area, and retrieving the filter element post-treatment requires multiple catheters to be inserted, advanced through the vasculature, and removed through the vasculature. This increases the length of time required for treatment, and may present a danger to the patient because the catheters must be advanced through narrow and/or tortuous areas of the vasculature, increasing the chance that the catheter will become snagged or cause damage to the vasculature.

### SUMMARY OF THE INVENTION

Exemplary embodiments of the present invention address the above disadvantages and other disadvantages not described above. Also, the present invention is not required to overcome the disadvantages described above.

An object of the present invention is to provide a treatment catheter which may also retrieve a deployed filter element thus decreasing the number of times the catheter is advanced through the vasculature.

Another object of the present invention is to provide a catheter which may use numerous different types and designs of filter elements.

According to an aspect of the present invention, there is provided a removable retrieval tip for use with a catheter, the removable retrieval tip including a cylindrical body; at least two protrusions which are located at a proximal end of the cylindrical body and extend radially outward from the cylindrical body; and a tapered portion located at a distal end of the cylindrical body.

According to another aspect of the present invention, there is provided a catheter tip for receiving a removable retrieval tip, the catheter tip including a catheter body having an inside wall and an outside wall; an inflatable balloon disposed on the outside wall of the catheter body; and at least two indentations which are spaced from a distal end of the catheter tip and are formed in the inside wall of the catheter body.

According to yet another aspect of the present invention, there is provided a catheter including a catheter tip at a distal end of the catheter, and a removable retrieval tip for insertion into the catheter tip, wherein the removable retrieval tip comprises a cylindrical body; at least two protrusions which are located at a proximal end of the cylindrical body and extend radially outward from the cylindrical body; and a tapered portion located at a distal end of the cylindrical body, and wherein the catheter tip comprises a catheter body having an inside wall and an outside wall; an inflatable balloon disposed on the outside wall of the cylindrical body; and at least two indentations which are spaced from a distal end of the catheter tip and are formed in the inside wall of the catheter body.

### BRIEF DESCRIPTION OF THE DRAWINGS

The above and other aspects of the present invention will become more apparent by describing in detail exemplary embodiments thereof with reference to the attached drawings, in which:

FIG. 1 is a view showing a balloon catheter according to an exemplary embodiment of the present invention;

FIG. 2 is cross-sectional view showing the distal end of the catheter of FIG. 1 along with a removable tip according to an exemplary embodiment of the present invention;

FIGS. 3A and 3B are a close-up cross-sectional view of the distal end of the catheter shown in FIG. 2 and a front view of the distal end of the catheter, respectively, according to an exemplary embodiment of the present invention;

FIGS. 4A and 4B are a close-up cross sectional view of the removable tip shown in FIG. 2 and a front view of the distal end of the removable tip, respectively, according to an exemplary embodiment of the present invention;

FIGS. 5A to 5C are views showing different arrangements of protrusions according to exemplary embodiments of the present invention; and

FIG. 6 is a cross-section view showing the distal end of the catheter of FIG. 1 along with a removable tip according to another exemplary embodiment of the present invention.

### DESCRIPTION OF EXEMPLARY EMBODIMENTS OF THE PRESENT INVENTION

Hereinafter, exemplary embodiments of the present inventive concept will be described in detail with reference to the drawings. The same reference numbers are used to denote the same elements in different drawings.

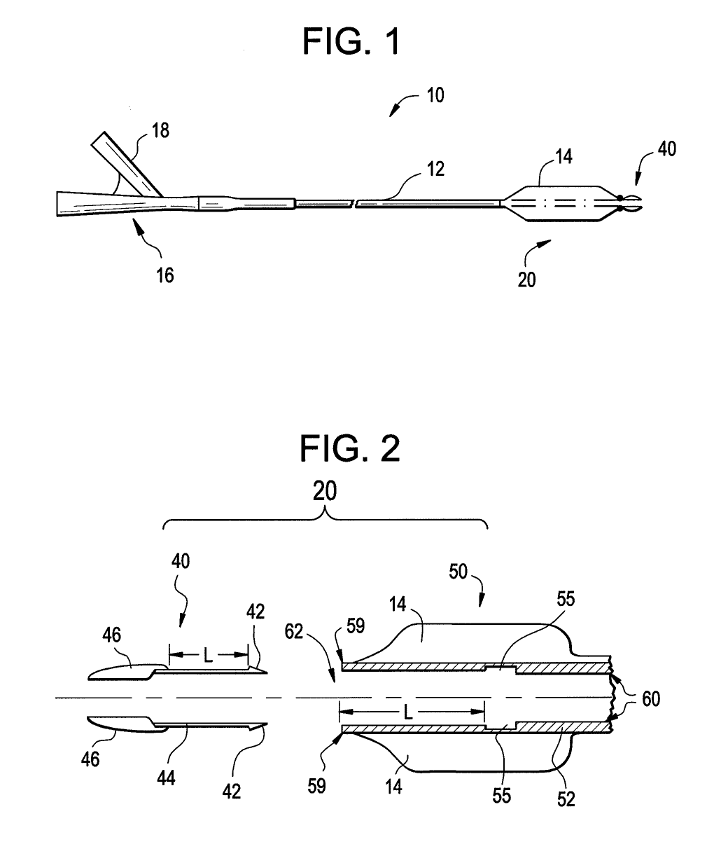

Referring to FIG. 1, a balloon catheter according to an exemplary embodiment is shown. The balloon catheter 10 includes a catheter body 12, having a distal end 20 and a proximal end 16. The catheter body 12 is flexible such that the catheter body 12 may be easily threaded through a vasculature of a patient. An inflatable balloon 14 is provided at the distal end 20 of the balloon catheter 10, and a balloon actuator 18 is provided at the proximal end 16 of the balloon catheter 10. A removable tip 40 is inserted into the balloon catheter 10 at the distal end of the inflatable balloon 14. The removable tip 40 is shown fully inserted into the balloon catheter 10 at the distal end of the inflatable balloon 14 in FIG. 1. However, the removable tip 40 is fully detachable from the balloon catheter 10, as will be described further below.

The following description assumes that a filter element has already been deployed according to a method known in the related art. U.S. Patent Application Publication No. 2002/0049467, the disclosure of which is herein incorporated by reference in its entirety, describes an example of a deployment method.

In use, the inflatable balloon 14 is deflated and the distal end 20 of the balloon catheter 10, having the removable tip 40 secured therein, is threaded, typically along a guidewire (not shown), into the upper leg of a patient to a treatment area, such that the inflatable balloon 14 is in position at an area at which treatment is desired. The previously deployed filter element is in place downstream of the treatment area. The inflatable balloon 14 is then inflated using the balloon actuator 18 located at the proximal end 16 of the balloon catheter 10, using a method which is generally known.

The inflatable balloon 14 is then deflated. During inflation and deflation of the inflatable balloon 14 to treat the occluded area, any plaque or other occlusion material that is dislodged during treatment is filtered by the filter element. The removable tip 40 at the distal end of the inflatable balloon 14, which is now deflated, is advanced to a proximal end of the previously deployed filter element. The filter element is then retrieved into a reception space in the distal end of the balloon catheter 10, and the balloon catheter 10, along with the retrieved filter element, is removed from the patient.

Turning now to FIG. 2, an enlarged, close-up cross-sectional view of the distal end 20 of the balloon catheter 10 is provided. The distal end 20 of the balloon catheter 10 includes a removable retrieval tip 40 and a catheter tip 50. FIG. 2 shows the removable tip 40 disengaged and removed from the catheter tip 50.

The removable retrieval tip 40 includes a cylindrical body 44. Protrusions 42 are located at the proximal end of the cylindrical body 44. The distal end of the cylindrical body 44 is tapered into a tapered portion 46 to allow for the balloon catheter 10 to more easily advance through the tortuous passages of a patient's vasculature.

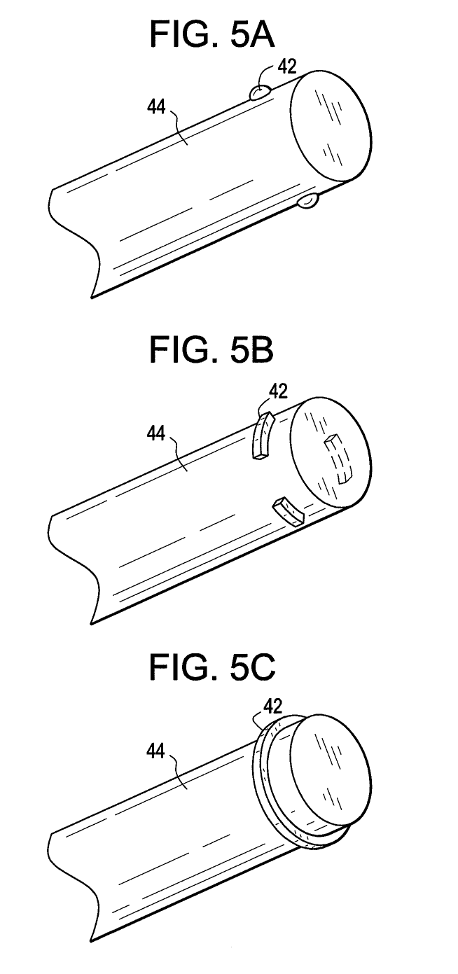

The protrusions 42 provided at the proximal end of the removable retrieval tip 40 may be of an arrow-shaped design as shown in FIG. 2, or may be hemispherical, as shown in FIG. 5A. Moreover, at least two protrusions 42 are provided. The protrusions 42 may also be provided in the form of a plurality of ridges, as shown in FIG. 5B. Lastly, the protrusions 42 may take the form of a thin cylindrical ridge, extending around the outer circumference of the cylindrical body 44, as shown in FIG. 5C. The protrusions 42 are spaced a distance L from the proximal end of the tapered portion 46 of the removable retrieval tip 40.

The catheter tip 50 includes a catheter body 52 having a distal end 59 and a proximal end 60 thereof. The inflatable balloon 14 is provided on an outside wall of catheter body 52 between the distal end 59 and the proximal end 60 of the catheter tip 50 at a distal end 20 of the balloon catheter 10. Indentations 55 are formed in the inside wall of the catheter body 52, and are spaced a distance L from the distal end 59 of the catheter tip 50. A reception space 62 for receiving a filter element is provided within the catheter body 52.

The indentations 55 provided in the catheter body 52 correspond to the geometry and location of the protrusions of the removable retrieval tip 40. Thus, there are at least two indentations 55.

In use, the removable retrieval tip 40 is inserted into the catheter tip 50 such that the protrusions 42 mate with the indentations 55 in order to secure, or lock, the removable retrieval tip 40 in place within the catheter tip 50. Thus, the removable retrieval tip 40 is prevented from disengaging while the balloon catheter 10 is inserted into the patient. Once thus engaged, the distal end 20 of the balloon catheter 10 is inserted into the patient in order to both provide treatment using the inflatable balloon 14 and to retrieve the previously deployed filter element, the process of which is described above.

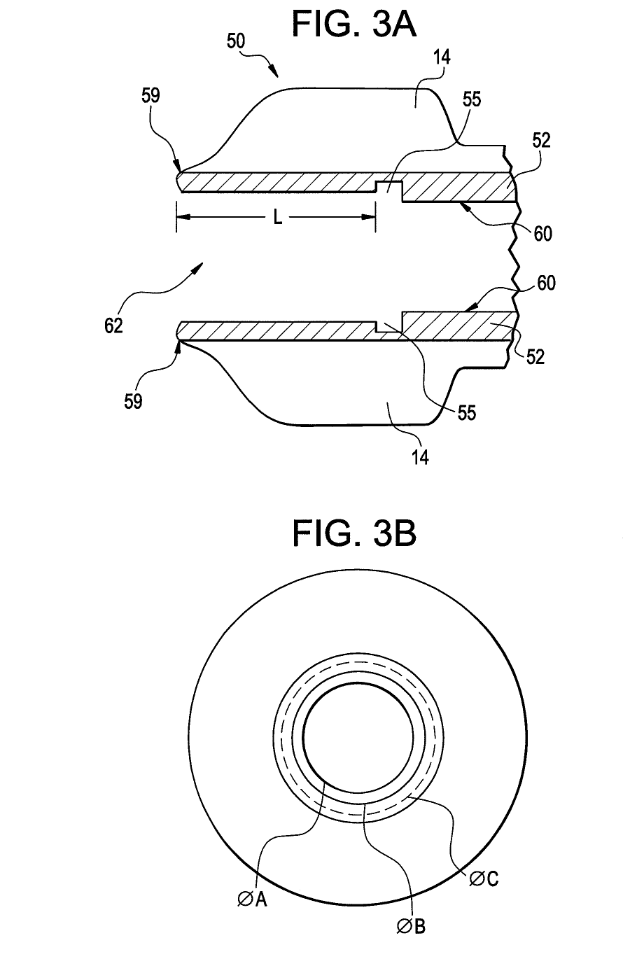

Turning now to FIGS. 3A and 3B, an enlarged, close-up sectional view of the catheter tip 50 is shown along with a front view of the catheter tip 50. As may be seen from FIG. 3B, the inner wall of at the proximal end 60 of the catheter tip 50 has a diameter A. The inner wall between the distal end 59 of the catheter tip 50 and the distal end of the indentations 55 has a diameter B, and the indentations 55 have a diameter C.

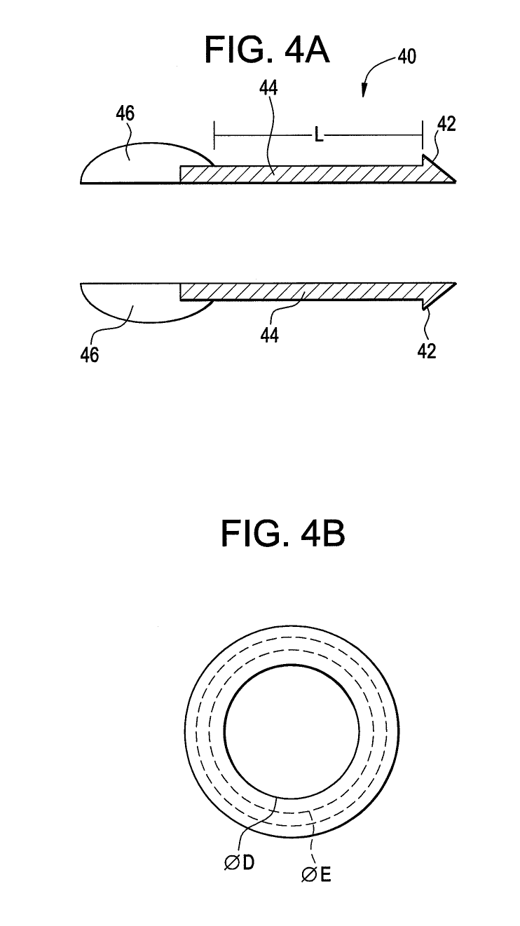

Turning to FIGS. 4A and 4B, an enlarged, close-up sectional view of the removable retrieval tip 40 is shown along with a front view of the removable retrieval tip 40. The inside wall of the cylindrical body 44 has a diameter D. The outside wall of the cylindrical body 44 has a diameter E, and the protrusion protrudes to a diameter F.

The inner diameter D of the removable retrieval tip 40 may be selected so that the removable retrieval catheter tip 40 may easily accommodate different sized and shaped filter elements. It is advantageous if the difference between diameters B and C be equal to the difference between the diameters E and F so that the protrusions 42 mate snugly with the indentations 55. Moreover, it is advantageous to select the diameters A, B, and C of the catheter tip 50 such that the diameter D of the inner wall of the removable retrieval tip 40 and the diameter A of the inner wall at the distal end 60 of the catheter tip 50 are equal so as to provide a smooth transition from the reception space 62 into the balloon catheter 10.

In use, it is contemplated that a physician would have multiple retrieval catheter tips 40 sized to accommodate various different filter elements. Thus, the physician would select a proper filter element for the treatment task, and then select a removable retrieval tip 40 which corresponds to the selected filter element. The selected removable retrieval tip 40 is then snapped into the balloon catheter or other treatment catheter and treatment and removal of the filter element following treatment are then carried out as described above.

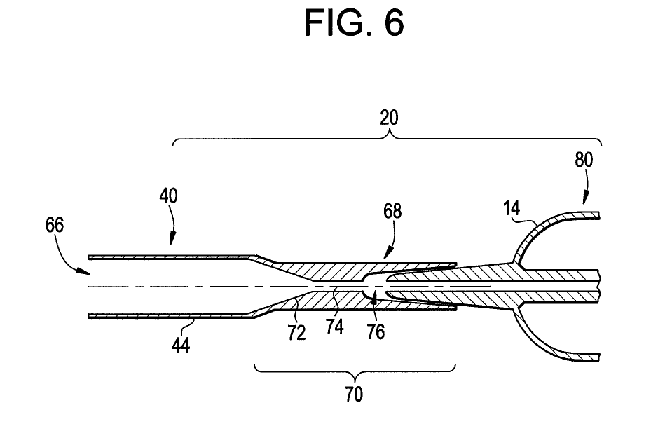

Turning now to FIG. 6, shown is a cross-section view showing the distal end of the catheter of FIG. 1 along with a removable tip according to another exemplary embodiment of the present invention. The distal end 20 of the catheter 10 of FIG. 1 includes a removable tip 40 and a catheter tip 80.

The catheter tip 80 is similar to the catheter tip 50 in the exemplary embodiment described above with reference to FIG. 2, except that no indentations 55 are provided. Accordingly, the catheter tip 80 in this exemplary embodiment may be any related art balloon catheter. The distal end of the catheter tip 80 is shown as slightly sloped in FIG. 6, and allows the related art balloon catheter to move more easily through a vasculature of a patient during treatment.

The removable tip 40 includes a cylindrical body, having a distal end 66 and a proximal end 68. The distal end 66 of the removable tip has a filter reception space for receiving a filter in order to collapse and retrieve the filter. At proximal end 68 of the removable tip 40 is formed an interface portion 70. The interface portion 70 includes a sloped region 72, a small bore region 74, and a balloon catheter tip receiving space 76.

The sloped region 72 of the interface portion 70 provides a transition from the diameter of the cylindrical body 44 of the removable tip 40 to a smaller diameter of the small bore region 74. At the distal end of the interface region 70, the sloped region 72 and the smaller diameter of the small bore region 74 act to prevent a filter, which is collapsed and retrieved into the reception space at the distal end of the removable tip 40, from sliding through the removable tip 40. At the proximal end of the interface region 70, the smaller diameter of the small bore region 74 and the balloon catheter tip receiving space 76 act to engage the distal end of the catheter tip 80.

In use, a filter element, which is secured to the distal end of a guidewire, is deployed as is commonly known in the art and as is described above. The removable tip 40 is then slid over the guidewire. Then, the catheter tip 80 is slid over the guidewire such that the catheter tip 80 interfaces with the removable tip 40 in the balloon catheter tip receiving space 76 of the interface portion 70 of the removable tip 40. Thus, the removable tip 40 and the catheter tip 80 are advanced to the treatment region, and treatment preformed by actuating the inflatable balloon 14 at the distal end of the catheter tip 80 by a method that is known in the art. Following treatment, the guidewire is pulled proximally such that the filter collapses and is recovered into the distal end of the removable tip 40. Once the filter is collapsed inside of the removable tip 40, the filter is pulled proximally such that a proximal end of the collapsed filter engages the sloped region 72 of the interface portion 70 within the removable tip 40. The sloped region 72 and/or the smaller diameter of the small bore region 74 then acts as an abutment or stop during retrieval, and stops the filter from continuing to slide proximally through the interface portion 70 of the removable tip 40. As the guidewire continues to be pulled proximally, the removable tip 40, with the filter collapsed and retrieved therein, and the catheter tip 80 of the balloon catheter 10 are withdrawn from the patient.

Accordingly, the exemplary embodiment of the present invention shown in FIG. 6 has an advantage in that no protrusions 42 or indentations 55 are used in order to secure or lock the removable tip to the catheter tip. In other words, the balloon catheter itself acts as an abutment or stop during retrieval, so that no active engagement is used at the interface between the tip and the balloon catheter. Alternatively, the removable tip may be provided with a softer material at a proximal end of the removable tip in order to provide a taper lock on the distal tip of the balloon catheter in order to actively engage the catheter with the removable tip.

Another advantage of the exemplary embodiment of the present invention shown in FIG. 6 is that a conventional “off the shelf” balloon catheter may be used with the removable tip.

The foregoing exemplary embodiments and advantages are merely exemplary and are not to be construed as limiting the present invention. The present teaching can be readily applied to other types of apparatuses. Also, the description of the exemplary embodiments of the present invention is intended to be illustrative, and not to limit the scope of the claims, and many alternatives, modifications, and variations will be apparent to those skilled in the art.

What is claimed is:

1. A removable retrieval tip for use with a catheter, the removable retrieval tip comprising:
a cylindrical body, having a reception space at a distal end thereof for receiving a filter;
a means, provided at a proximal end of the cylindrical body, for interfacing with the catheter.

2. The removable retrieval tip of claim 1, wherein the means for interfacing with the catheter receives a tip of the catheter such that the catheter acts as a shaft for the removable retrieval catheter tip.

3. The removable retrieval tip of claim 1, where in the means for interfacing with the catheter acts as an abutment to limit proximal movement of the removable retrieval tip over the tip of the catheter during retrieval of a filter element.

4. The removable retrieval tip of claim 3, wherein the means for interfacing with the catheter comprises a soft proximal portion to provide a taper lock on the tip of the catheter.

5. The removable retrieval tip of claim 1, further comprising a tapered portion located at a distal end of the cylindrical body

6. The removable retrieval tip of claim 1, wherein the means for interfacing with the catheter comprises at least two protrusions which are located at a proximal end of the cylindrical body and extend radially outward from the cylindrical body.

7. The removable retrieval tip of claim 6, wherein the at least two protrusions are equally spaced around the cylindrical body.

8. The removable retrieval tip of claim 6, wherein the at least two protrusions form ridges on an outside wall of the cylindrical body.

9. The removable retrieval tip of claim 8, wherein the ridges extend around a full circumference of the cylindrical body.

10. The removable retrieval tip of claim 6, wherein the protrusions are hemispherical in shape or are arrow-shaped.

11. A catheter tip for receiving a removable retrieval tip, the catheter tip comprising:
a catheter body having an inside wall and an outside wall;
an inflatable balloon disposed on the outside wall of the catheter body; and
at least two indentations which are spaced from a distal end of the catheter tip and are formed in the inside wall of the catheter body.

12. The catheter tip of claim 7, wherein the at least two indentations formed in the inside wall of the catheter body are spaced a distance from the distal end of the catheter tip.

13. A system for retrieval of an embolic filter device, the system comprising:
a catheter having a catheter tip at a distal end thereof, and
a removable retrieval tip comprising:
a cylindrical body; and
an interface portion provided at a proximal end of the cylindrical body for interfacing with the catheter tip

14. The removable retrieval tip of claim 13, wherein the interface portion receives a tip of the catheter such that the catheter acts as a shaft for the removable retrieval catheter tip.

15. The removable retrieval tip of claim 13, where in the interface portion acts as an abutment to limit proximal movement of the removable retrieval tip over the tip of the catheter during retrieval of a filter element.

16. The removable retrieval tip of claim 15, wherein the interface portion comprises a soft proximal portion to provide a taper lock on the tip of the catheter.

17. The removable retrieval tip of claim 13, further comprising a tapered portion located at a distal end of the cylindrical body.

18. The removable retrieval tip of claim 13, wherein the interface portion comprises at least two protrusions which are located at a proximal end of the cylindrical body and extend radially outward from the cylindrical body; and
wherein the catheter tip comprises:
a catheter body having an inside wall and an outside wall;
an inflatable balloon disposed on the outside wall of the catheter body; and
at least two indentations which are spaced from a distal end of the catheter tip and are formed in the inside wall of the catheter body.

19. The catheter of claim 18, wherein the at least two indentations formed in the inside wall of the catheter body correspond to the at least two protrusions of the cylindrical body.

20. A removable retrieval tip for use with a catheter, the removable retrieval tip comprising:
a filter reception space for receiving a filter; and
an interface portion for interfacing with the catheter.

21. The removable retrieval tip of claim 20, wherein the interface portion has a sloped region at a distal end thereof and tip receiving space at a proximal end thereof, and a wall thickness in a region between the sloped region and the tip receiving space is thicker than a wall thickness of the filter reception space such that the filter is prohibited from passing through the region between the sloped region and the receiving space.
